(19) United States
(12) Patent Application Publication
(10) Pub. No.: US 2023/0372389 A1
(43) Pub. Date: Nov. 23, 2023

(54) INHALATION FORMULATION, AND PREPARATION METHOD THEREFOR AND APPLICATION THEREOF

(71) Applicant: Zhi LIU, Jiangsu (CN)

(72) Inventors: Zhi LIU, Jiangsu (CN); Xin LIU, Jiangsu (CN)

(21) Appl. No.: 18/248,056

(22) PCT Filed: Sep. 30, 2021
(86) PCT No.: PCT/CN2021/122210

§ 371 Date: Apr. 6, 2023

(30) Foreign Application Priority Data
Oct. 9, 2020 (CN) 202011073726.X

(51) Int. Cl. A61K 33/14; A61K 9/00; A61K 47/26; A61K 33/00; A61P 9/12

(52) U.S. Cl. CPC A61K 33/14; A61K 9/0078; A61K 9/0075; A61K 47/26; A61K 33/00; A61P 9/12

(57) ABSTRACT

An inhalation formulation, and a preparation method therefor and an application thereof. The inhalation formulation can be presented in the form of (1) a liquid inhalation formulation comprising a potassium salt and water or (2) a dry powder inhalation formulation comprising a potassium salt and a drug carrier. The inhalation formulation can be formulated for oral or respiratory tract administration, effectively supplement potassium when the potassium level is low, and change the local sodium-potassium balance of the oral mucosa or the mucosa of the respiratory tract to achieve a beneficial physiological effect.

The present application claims the priority of Chinese patent application 202011073726X filed on Oct. 9, 2020. The contents of the Chinese patent application are incorporated herein by reference in their entireties.

### TECHNICAL FIELD

The present disclosure relates to an inhalation formulation, and a preparation method therefor and an application thereof.

### BACKGROUND

The content of potassium in human body (the atomic number of potassium is 19, and the relative atomic weight is 39.1) is second only to calcium and phosphorus, and is twice as high as that of sodium (the relative atomic weight is 23). Normal human body contains about 140-150 g of potassium (i.e. 3.6 to 3.8 mol). About 98% of the potassium ions in the human body exist in the intracellular fluid, which is the main cation of the intracellular fluid. The concentration of potassium ions in the cell is about 30 times higher than that outside the cell (about 100-150 mmol/L). The sodium ion mainly exists outside the cell, and its concentration outside the cell is about 10 times higher than that inside the cell. The sodium concentration in plasma is much higher than the potassium concentration (serum potassium concentration ranges from 3.5 to 5 mmol/L, while serum sodium is 140 mmol/L), but the potassium concentration in muscle tissue and milk is several times higher than sodium. This huge difference in concentrations of potassium and sodium ions inside and outside the cell creates a potential difference across the cell membrane. To maintain the condition of high extracellular sodium and low extracellular potassium, and high intracellular potassium and low intracellular sodium, the cells must constantly “pump” the intracellular sodium ions out and “pump” the extracellular potassium ions in.

Since this transmembrane ion transport is against the concentration gradient of ions (that is, to transfer ions from a place of low concentration to a place of high concentration), these processes require energy. On average, about 20% of the energy consumed by cells is used to maintain the imbalance of potassium and sodium ions inside and outside the cells, and the energy used by nerve cells for this purpose can account for 60% of the total energy consumption of nerve cells. Even if one is sitting there doing nothing, the cells in the body are constantly pumping potassium in and sodium out. Animal cells have a difference in transmembrane potential, which is positive extracellularly and negative intracellularly, with an amplitude of about −60 mV to −150 mV. This is related to the imbalance of intracellular and extracellular concentrations of many ions, such as potassium ions and chloride ions, but the difference in transmembrane potential is mainly formed by extracellular positively charged sodium ions. The high concentration of extracellular sodium ions is also a way to store energy. It is the same as the hydrogen ions outside the cell membrane, like the water stored in the reservoir, and the water from a higher level has potential energy. In human body, sodium ions also regulate blood volume and blood pressure, so sodium ions are needed by human body, which is why people need to add sodium chloride to their daily diet. However, the most important role of extracellular sodium ions in animals is to generate nerve impulses. Potassium plays an important role in maintaining the normal function of heart and in the metabolism of cells; potassium contributes to the normal operation of nerve conduction function, and it works together with sodium to adjust pH of fluids in body; potassium deficiency or excess potassium in human body may affect health.

At present, potassium ions are supplemented through oral or intravenous administration, and potassium ions enter the circulatory system and increase the concentration of potassium ions in body fluids as one of the means of lowering blood pressure. The basis of this therapy is that potassium ions enter the circulatory system to increase the potassium ion concentration of body fluids, which requires a relatively large amount of potassium ion supplementation and absorption to achieve, with the fact considered that the total body fluids of normal adults account for about 60% of body weight. At present, potassium supplementation adopts a treatment method of controlling the total amount, supplementing in batches and observing while treating. Those who can take potassium supplements orally can take them orally, with a single dose of 3-7.5 g, generally no more than 15 g per day (in terms of potassium chloride, molecular weight 74.55, about 200 mmol). The concentration of the oral solution is 1.268% (in terms of potassium chloride, percentage by weight), but the total amount of potassium chloride required for oral administration of solid is the same as that of the liquid. There is a limit to the speed of intravenous potassium supplementation. The potassium content per liter of infusion should not exceed 40 mmol (equivalent to 3 g of potassium chloride). The infusion should be dripped slowly, and the amount of potassium input should be controlled below 10-20 mmol/h (equivalent to 0.75-1.5 g/h of potassium chloride). The total amount of intravenous potassium supplementation should not be too much. Potassium supplementation should be based on the blood potassium level. A single potassium supplementation is 4.5 to 6 g, generally no more than 15 g per day (in terms of potassium chloride).

However, both oral and intravenous potassium supplementation have safety and efficacy issues. For example, when intravenous infusion is used for potassium supplementation, the tunica intima of vein is usually irritated due to high concentration of the liquid medicine, too fast infusion rate and thin human vein, which causes pain. If potassium is supplemented by intravenous infusion, due to the rapid infusion rate and damage to the patient's renal function, special attention should be paid to whether the patient has hyperkalemia. Once such symptoms appear, it is necessary to deal with them in time. Taking potassium chloride orally can irritate the stomach, causing stomach pain. Therefore, potassium chloride generally needs to be taken after meals. Potassium that is absorbed orally or entered into the body through veins also needs to be excreted through the kidneys to maintain the relative balance in the body.

In a related art, physiological saline or sea water is used to clean mucosa of nasal cavity or respiratory tract. However, whether in physiological saline or water from natural sources, the concentration of sodium salts is much higher than that of potassium salts. Almost all liquid water on earth contains significantly more sodium than potassium. Whether it is river water, lake water or sea water, sodium content is much more than potassium. For example, the concentration of sodium in seawater is 47 times that of potassium (sodium 470 mmol/L (mM), potassium 10 mM). The salinity in river water varies with rivers, but generally the concentration of sodium is 10 times that of potassium (sodium is about 0.4 mM, potassium approximately 0.04 mM). Although the amount of potassium in the earth's crust is actually similar with that of sodium: 2.8% sodium and 2.6% potassium. Therefore, the concentration of sodium ions in natural seawater, artificially prepared physiological saline or other saline solutions (such as lactated Ringer's solution, etc.) used for nasal cleaning is much higher than that of potassium ions. This approach does not increase the potassium/sodium ion ratio in mucosa of nasal cavity or respiratory tract (Reference: Science Network https://blog.csdn.net/cf2SudS8x8F0v/article/details/84038809 “More potassium and less sodium in cells—the third largest vestige of primitive organisms”, author Zhu Qinshi).

### CONTENT OF THE PRESENT INVENTION

In order to overcome the above-mentioned defects of oral and intravenous potassium supplementation, the present disclosure provides an inhalation formulation, and a preparation method therefor and an application thereof. The inhalation formulation can be administered through the oral cavity or the respiratory tract, effectively replenish potassium in mucosa of the oral cavity or respiratory tract at a low dose and also change the local sodium and potassium balance of mucosa of the oral cavity or respiratory tract, bringing beneficial physiological effects.

In order to achieve the above-mentioned object, the present disclosure adopts the following technical solutions:

The present disclosure provides an inhalation formulation comprising two forms:
(i) a liquid inhalation formulation comprising a potassium salt and water, with the molar concentration of potassium being 0.001 to 27,410 mmol/L; or
(ii) a dry powder inhalation formulation, which comprises a potassium salt and a drug carrier, the content of the potassium salt is 0.01% to 100%, and the percentage is the mass percentage of the potassium salt in the dry powder inhalation formulation.

In the present disclosure, the potassium salt may be the one conventionally used in the art, preferably comprises one or more selected from a group consisting of potassium chloride, potassium acetate, potassium gluconate, potassium aspartate, potassium amino acid complex, potassium carbonate, bicarbonate potassium, potassium citrate, potassium dihydrogen citrate, potassium hydrogen citrate, potassium tartrate, potassium hydrogen tartrate, potassium hydrogen tartrate, potassium hydroxide, potassium phosphate, potassium hydrogen phosphate, potassium dihydrogen phosphate, potassium sulfate, potassium bisulfate, potassium nitrate, potassium alginate and potassium sorbate; more preferably potassium chloride, potassium acetate, potassium carbonate, potassium bicarbonate, potassium tartrate, potassium hydrogen tartrate, potassium hydrogen tartrate, potassium citrate, or potassium hydrogen citrate.

In the present disclosure, the molar concentration of potassium in the liquid inhalation formulation may be 0.001 to 10,000 mmol/L, preferably 0.001 to 4,700 mmol/L, preferably 3 to 3,500 mmol/L, more preferably 10 to 2,000 mmol/L, more preferably 20 to 1,500 mmol/L, further preferably 50 to 1,000 mmol/L, even more preferably 75 to 700 mmol/L. The molar concentration of potassium in the liquid inhalation formulation is, for example, 25.5 mmol/L, 154 mmol/L, 537 mmol/L or 1,221 mmol/L.

In the present disclosure, the molar concentration of potassium in the liquid inhalation formulation is preferably 0.001 to 2,500 mmol/L, preferably 0.001 to 1,500 mmol/L, more preferably 3 to 1,000 mmol/L, and more preferably 20 to 400 mmol/L, or even more preferably 50 to 200 mmol/L.

In the present disclosure, in the liquid inhalation formulation, the potassium salt is potassium chloride, and the molar concentration of the potassium chloride can be 0.001 to 4,700 mmol/L, for example, 25.5 mmol/L, 154 mmol/L, 537 mmol/L or 1,221 mmol/L.

In the present disclosure, in the liquid inhalation formulation, the potassium salt is potassium acetate, and the molar concentration of the potassium acetate may be 0.001 to 27,410 mmol/L, for example, 10,000 mmol/L.

In the present disclosure, the pH of the liquid inhalation formulation may be 3.0 to 9.0, preferably 4.0 to 8.0, more preferably 5.0 to 7.0, further preferably 5.5 to 6.5.

In the present disclosure, the liquid inhalation formulation may also contain conventional pharmaceutically acceptable additives in the art. The additives preferably comprise one or more selected from a group consisting of a diluent, an acidity regulator, an isotonic regulator and a preservative.

Herein, the diluent can be a conventional diluent in the art, preferably one or more selected from a group consisting of ethanol, isopropanol, propylene glycol, glycerin, mannitol, lactose, gelatin, starch, pregelatinized starch, dextrin, cyclodextrin, sucrose, taurine, amino acid, microcrystalline cellulose, calcium sulfate, calcium hydrogen phosphate, magnesium oxide, calcium carbonate, hydroxypropyl methylcellulose, povidone, starch slurry, syrup, sodium carboxymethyl starch and crospovidone.

The acidity regulator, also known as pH controller, can be a pharmaceutical additive conventionally used in the art to adjust or maintain the pH value. The acidity regulator can be acid, alkali, neutralizing agent or buffer, preferably one or more selected from a group consisting of meglumine, boric acid, sodium acetate anhydrous, sodium acetate trihydrate, crystalline sodium acetate, tromethamine, acetic acid, amino acid, hydrochloric acid, phosphoric acid, ammonium chloride, sodium citrate, dilute ammonia solution, calcium acetate, anhydrous disodium hydrogen phosphate, anhydrous sodium carbonate, potassium bicarbonate, ammonium acetate, acetic acid, tartaric acid, methanesulfonic acid, and succinate acids. The content of the acidity regulator can be 0.05% to 20%, and the percentage is by weight.

Herein, the isotonic regulator may be a conventional non-sodium salt osmotic pressure regulator in the art, preferably one or more selected from a group consisting of glucose, sucrose, glycerin and xylitol. The content of the isotonic regulator can be 0.01% to 20%, and the percentage is by weight.

Herein, the preservative can be a conventional pharmaceutical preservative in the art, preferably phenol substitutes (such as biphenol, cresol or xylenol), cationic surface active substances, halogens, oxidizing agents (such as hydrogen peroxide or permanganate), aniline dyes, acridine dyes, heavy metal salts, alcohols or aldehydes. The preservative may also be a food preservative such as benzoic acid, sodium benzoate, sorbic acid, potassium sorbate or calcium propionate. The content of the preservative may be 0.01% to 20%, and the percentage is by weight.

In the present disclosure, the liquid inhalation formulation is preferably potassium chloride aqueous solution, potassium acetate aqueous solution, potassium carbonate aqueous solution, potassium bicarbonate aqueous solution, potassium tartrate aqueous solution, potassium hydrogen tartrate aqueous solution, potassium citrate aqueous solution or potassium hydrogen citrate aqueous solution.

In the present disclosure, the content of the potassium salt in the dry powder inhalation formulation is preferably 1% to 90%, more preferably 1% to 50%, and more preferably 1% to 20%.

In the present disclosure, the drug carrier of the dry powder inhalation formulation can be one or more selected from a group consisting of mannitol, lactose, gelatin, starch, amino acid, pregelatinized starch, dextrin, cyclodextrin, sucrose, microcrystalline cellulose, calcium sulfate, calcium hydrogen phosphate, magnesium oxide, calcium carbonate, hydroxypropyl methylcellulose (HPMC), povidone (PVP), starch slurry, sugar syrup, sodium carboxymethyl starch and crospovidone.

In the present disclosure, the dry powder inhalation formulation preferably comprises potassium chloride and lactose. Herein, the content of described potassium chloride is preferably 2% to 10%, more preferably 2% to 6%; for example, 2.2%, 5.7% or 9.3%, the percentage is the mass percentage of the potassium chloride in the dry powder inhalation formulation.

In the present disclosure, when the content of the potassium salt in the dry powder inhalation formulation is 100%, the dry powder inhalation formulation is pure potassium salt, such as pure potassium chloride.

In the present disclosure, the average particle size of the dry powder inhalation formulation may be 0.05 to 1,000 μm, preferably 0.1 to 500 μm, more preferably 0.2 to 250 μm, such as 74 μm; or 0.2 to 5 μm, preferably 0.5 to 2 μm, such as 1 μm or 4 μm; or 5 to 10 μm, such as 7 μm; or 10 to 20 μm, such as 15 μm. Particles with an average particle size of 10 to 20 μm can be deposited on the nasal mucosa, and particles with an average particle size of 5 to 10 μm can enter the upper respiratory tract, and particles with an average particle size of less than 2 μm can directly enter the lungs through the mucosa.

The present disclosure also provides a preparation method for the inhalation formulation, which comprises uniformly mixing the components.

When the inhalation formulation is a liquid inhalation formulation, the preparation method comprises uniformly mixing the components to form a solution.

When the inhalation formulation is a dry powder inhalation formulation, the preparation method comprises uniformly mixing the components to form a solid mixture with a specific particle size or potassium salt particles with a specific particle size.

In the present disclosure, a preferred preparation method for the dry powder inhalation formulation comprises: firstly dissolving the potassium salt in water, then mixing the obtained aqueous solution of the potassium salt with a drug carrier, drying, and then making powder.

In a preferred embodiment of the present disclosure, the preparation method for the dry powder inhalation formulation comprises: firstly dissolving potassium chloride in water, then adding the obtained potassium chloride aqueous solution into lactose by spray-drying, and then making powder.

The present disclosure also provides an administration method comprising applying the inhalation formulation to the oral cavity or respiratory tract. The respiratory tract comprises nasal cavity, pharynx, larynx, trachea, bronchi and branches of bronchi at all levels within the lungs. The section of the respiratory tract from the nose to the larynx is called the upper respiratory tract. The trachea, bronchi and branches of bronchi at all levels within the lungs are the lower respiratory tract.

In the present disclosure, the application method for the liquid inhalation formulation may comprise spraying, dripping or washing; preferably spraying.

Herein, the spraying can be carried out using a conventional spraying device in the art, such as a push-type manual nebulizer, an electric spraying device, a pneumatic spraying device, a mechanical spraying device or an ultrasonic atomizer. The spray device can be quantitative or non-quantitative. The liquid inhalation formulation can be dispersed into fine mist droplets by the spray device, which was sprayed out in the form of aerosol and suspended in the gas.

The average particle size of the aerosol may be 0.05 to 1,000 μm, preferably 0.1 to 500 μm, more preferably 0.2 to 250 μm, such as 74 μm; or 0.2 to 4 μm, such as 1 μm; or 5 to 10 μm, such as 7 μm; or 10 to 20 μm, such as 15 μm. Aerosol with an average particle size of 10 to 20 μm can be deposited on the nasal mucosa, and aerosol with an average particle size of 5 to 10 μm can enter the upper respiratory tract, and aerosol with an average particle size of less than 2 μm can directly enter the lungs through the mucosa.

In the present disclosure, the single dose of the liquid inhalation formulation may be 0.01 to 0.5 mmol in terms of potassium ions. When the potassium salt is potassium chloride, in terms of potassium chloride, the single dose of the liquid inhalation formulation is preferably 0.74 to 37.3 mg, or 0.74 to 140 mg.

In the present disclosure, the application method for the dry powder inhalation formulation may be direct inhalation or a dry powder inhalation device. Herein, the dry powder inhalation device can be conventional in the art, preferably a quantitative pressure inhalation device or a dry powder atomization inhalation device.

In the present disclosure, the single dose of the liquid inhalation formulation may be 0.01 to 0.5 mmol in terms of potassium ions. When the potassium salt is potassium chloride, in terms of potassium chloride, the single dose of the dry powder inhalation formulation is preferably 0.74 to 37.3 mg, or 0.74 to 140 mg.

Since potassium salt has a very high safe dose, the supplementation of potassium ion may also be achieved continuously through breathing by the human body with continuous liquid atomization or dry powder spray in the environment. The upper limit should not exceed that of oral or intravenous potassium supplementation, with a single dose of approximately 3 g, generally no more than 15 g per day (in terms of potassium chloride). Multiple doses may be employed.

The present disclosure also provides a use of the inhalation formulation in the preparation of medicine for treating hypertension.

The present disclosure also provides a use of the inhalation formulation in the preparation of sedative medicine.

The above-mentioned preferred conditions can be combined arbitrarily to obtain preferred embodiments of the present disclosure in accordance with common knowledge in the art.

The reagents and raw materials used in the present disclosure are all commercially available.

The positive progress effect of the present disclosure is:

In the present disclosure, the administration is achieved by inhalation through the oral cavity or respiratory tract, providing low-dose potassium ions, and the potassium ions can enter the blood system through the mucoca, which increases the local potassium ion concentration of the mucosa of oral or respiratory tract, especially the local potassium/sodium ion ratio of the mucosa of oral or respiratory tract, under the condition that the dosage and blood drug concentration are far lower than those of other existing routes of administration (oral or intravenous injection), and thereby reduces the resistance of the cell membrane to transport potassium ions, reduces energy consumption and effectively supplements potassium. The unexpectedness of the present disclosure is to use a small amount of potassium ions to act on the mucosa of oral cavity or respiratory tract, change the local sodium-potassium balance of the mucosa, activate certain cells in the mucosa layer, and thus have unexpected beneficial physiological effects (such as lowering blood pressure and playing a role in sedation, etc.).

### BRIEF DESCRIPTION OF THE DRAWINGS

FIG. 1 is the blood pressure record of a subject in Effect Example 5.

FIG. 2 is the blood pressure record of a subject in Effect Example 6.

FIG. 3 is the blood pressure record of a subject in Effect Example 7.

FIG. 4 is the blood pressure record of a subject in Effect Example 8.

### DETAILED DESCRIPTION OF THE PREFERRED EMBODIMENT

The following examples further illustrate the present disclosure, but the present disclosure is not limited thereto. Experimental methods without specific conditions in the following embodiments are selected according to conventional methods and conditions, or according to the commercial specification.

### Embodiment 1

2.3 g of KCl (molecular weight 74.55) was dissolved in 200 mL of purified water (molar concentration 154 mmol/L, or 1.15% w/v) to form a clear solution, that is, a liquid inhalation formulation; then the solution was dispensed in a 50 mL pump-type manual nebulizer.

When it is needed to be used, one may aim the nozzle at the left nostril while pressing and stopping the right nostril. While inhaling through the left nostril, one may press down on the piston to inhale the generated mist droplets into the nasal cavity. Similar operation can be used for the right nostril. The amount of liquid inhaled each time was about 0.2 mL, containing 2.3 mg of KCl (0.03 mmol KCl). In the case of using in both left and right nostrils, about 4.6 mg of KCl (0.06 mmol KCl) would be inhaled in a single dose. The inhaler did not experience any discomfort. Alternatively, when it is needed to be used, the liquid inhalation formulation is sprayed into the mouth and tongue, with a slightly bitter taste but no other discomfort.

### Embodiment 2

8 g of KCl was dissolved in 200 mL of purified water (molar concentration 537 mmol/L, or 4.0% w/v) to form a clear solution, that is, a liquid inhalation formulation; then the solution was dispensed in a 50 mL pump-type manual nebulizer.

When it is needed to be used, one may aim the nozzle at the left nostril while pressing and stopping the right nostril. While inhaling through the left nostril, one may press down on the piston to inhale the generated mist droplets into the nasal cavity. Similar operation can be used for the right nostril. The amount of liquid inhaled each time is about 0.2 mL, containing 8 mg of KCl (0.11 mmol KCl). In the case of using in both left and right nostrils, about 16 mg of KCl (0.22 mmol KCl) would be inhaled in a single dose. Due to the high concentration and osmotic pressure of the solution, the user had a slight dizziness, which basically disappeared after about 10 minutes.

### Embodiment 3

2.3 g of KCl was dissolved in 200 mL of purified water (molar concentration 154 mmol/L, or 1.15% w/v) to form a clear solution, that is, a liquid inhalation formulation; then the solution was dispensed in a commercially available ultrasonic nebulizer.

When it is needed to be used, the ultrasonic nebulizer is turned on and placed near the nasal cavity and oral cavity. A continuously-generated mist droplets with a particle size of less than 100 μm can benhaled into the respiratory tract. The ultrasonic nebulizer can nebulize 6 mL of liquid every 30 minutes, which is equivalent to 69 mg of KCl (0.9 mmol). About 10 minutes per inhalation, and about 23 mg of KCl (0.30 mmol KCl) was inhaled. The inhaler did not experience any discomfort.

### Embodiment 4

0.38 g of KCl (molecular weight 74.55) was dissolved in 200 mL of purified water (molar concentration 25.5 mmol/L, or 0.19% w/v) to form a clear solution, that is, a liquid inhalation formulation; then the solution was dispensed in a 50 mL pump-type manual nebulizer.

When it is needed to be used, one may aim the nozzle at the left nostril while pressing and stopping the right nostril. While inhaling through the left nostril, one may press down on the piston to inhale the generated mist droplets into the nasal cavity. Similar operation can be used for the right nostril. The amount of liquid inhaled each time was about 0.2 mL, containing 0.38 mg of KCl (0.005 mmol KCl). In the case of using both left and right nostrils, about 0.76 mg of KCl (0.01 mmol KCl) would be inhaled in a single dose. The inhaler did not experience any discomfort.

Alternatively, when it is needed to be used, the liquid inhalation formulation is sprayed into the mouth and tongue, with a slightly bitter taste but no other discomfort.

### Embodiment 5

18.2 g of KCl was dissolved in 200 mL of purified water (molar concentration 1,221 mmol/L, or 9.1% w/v) to form a clear solution, that is, a liquid inhalation formulation; then the solution was dispensed in a 50 mL pump-type manual nebulizer.

When it is needed to be used, one may aim the nozzle at the left nostril while pressing and stopping the right nostril. While inhaling through the left nostril, one may press down on the piston to inhale the generated mist droplets into the nasal cavity. Similar operation can be used for the right nostril. The amount of liquid inhaled each time was about 0.2 mL, containing 18.6 mg of KCl (0.25 mmol KCl). In the case of using both left and right nostrils, about 37.3 mg of KCl (0.5 mmol KCl) would be inhaled in a single dose. Due to the high concentration and osmotic pressure of the solution, the user had a slight dizziness, which basically disappeared after about 30 minutes.

### Embodiment 6

70 g of KCl was dissolved in 200 mL of purified water (molar concentration 4700 mmol/L, or 35% w/v) to form a clear solution, that is, a liquid inhalation formulation; then the solution was dispensed in a 50 mL pump-type manual nebulizer.

When it is needed to be used, one may aim the nozzle at the left nostril while pressing and stopping the right nostril. While inhaling through the left nostril, one may press down on the piston to inhale the generated mist droplets into the nasal cavity. Similar operation can be used for the right nostril. The amount of liquid inhaled each time was about 0.2 mL, containing 70 mg of KCl (0.94 mmol KCl). In the case of using both left and right nostrils, about 140 mg of KCl (1.88 mmol KCl) would be inhaled in a single dose. Due to the high concentration and osmotic pressure of the solution, the user had a slight dizziness, which basically disappeared after about 60 minutes.

The above-mentioned solution was added into a commercially available ultrasonic nebulizer to form a mist rich in potassium ions. The ultrasonic nebulizer can nebulize 6 mL of liquid every 30 minutes, which is equivalent to 2.1 g of KCl (28 mmol). About 2 minutes per inhalation, and about 140 mg of KCl (1.88 mmol KCl) was inhaled. The inhaler did not experience any discomfort. When inhaling through the nose or mouth, one felt basically no discomfort caused by the high concentration of electrolytes (hyperosmotic pressure) in the solution of the mist droplets.

### Embodiment 7

2 g of KCl solid was taken, grinded in a mill and passed through a 200-mesh sieve, and the particle size was less than 74 μm to obtain a dry powder inhalation formulation.

When it is needed to be used, 0.01 g of fine powder was taken, put on a clean white paper and placed under the left nostril. Press and stop the right nostril, and inhale forcefully, and then the above-mentioned powder can be partially inhaled into the nasal cavity. In the case of using both left and right nostrils, about 10 mg of KCl (0.134 mmol KCl) would be inhaled in a single dose. The inhaler did not experience any discomfort.

### Embodiment 8

2.3 g of KCl solid was taken, grinded in the mill and passed through a 200-mesh sieve, and the particle size was less than 74 μm; 100 g of medicinal lactose was taken and grinded in the mill and passed through a 200-mesh sieve, and the particle size was less than 74 μm. The obtained KCl powder was dissolved in 20 mL of purified water to form a clear solution; and the obtained KCl solution was added to the lactose by spraying. After drying, it was ground into a fine powder and passed through a 200-mesh sieve, and the particle size was less than 74 μm to obtain a dry powder inhalation formation, wherein the content of KCl was about 2.2%.

When it is needed to be used, 0.2 g of fine powder was taken, put on a clean white paper and placed under the left nostril. Press and stop the right nostril, and inhale forcefully, and then the above-mentioned powder can be partially inhaled into the nasal cavity. The amount of solid powder inhaled each time was about 0.2 g, containing 4.4 mg of KCl (0.06 mmol KCl). In the case of using both left and right nostrils, about 8.8 mg of KCl (0.12 mmol KCl) would be inhaled in a single dose. The inhaler did not experience any discomfort.

### Embodiment 9

6 g of KCl solid was taken, grinded in the mill and passed through a 200-mesh sieve, and the particle size was less than 74 μm; 100 g of medicinal lactose was taken and grinded in the mill and passed through a 200-mesh sieve, and the particle size was less than 74 μm. The obtained KCl powder was dissolved in 20 mL of purified water to form a clear solution; and the obtained KCl solution was added to the lactose by spraying. After drying, it was ground into a fine powder and passed through a 200-mesh sieve, and the particle size was less than 74 μm to obtain a dry powder inhalation formation, wherein the content of KCl was about 5.7%.

When it is needed to be used, 0.2 g of fine powder was taken, put on a clean white paper and placed under the left nostril. Press and stop the right nostril, and inhale forcefully, and then the above-mentioned powder can be partially inhaled into the nasal cavity. The amount of solid powder inhaled each time was about 0.2 g, containing 11.4 mg of KCl (0.153 mmol KCl). In the case of using both left and right nostrils, about 22.8 mg of KCl (0.306 mmol KCl) would be inhaled in a single dose. Due to the high salt content, it absorbed water and dissolved in the nasal mucosa after inhalation, and the salt concentration and osmotic pressure of the formed solution were high. The user had a slight dizziness, which basically disappeared after an hour.

### Embodiment 10

10.3 g of KCl solid was taken, grinded in the mill and passed through a 200-mesh sieve, and the particle size was less than 74 μm; 100 g of medicinal lactose was taken and grinded in the mill and passed through a 200-mesh sieve, and the particle size was less than 74 μm. The obtained KCl powder was dissolved in 50 mL of purified water to form a clear solution; and the obtained KCl solution was added to the lactose by spraying. After drying, it was ground into a fine powder and passed through a 200-mesh sieve, and the particle size was less than 74 μm to obtain a dry powder inhalation formation, wherein the content of KCl was about 9.3%.

When it is needed to be used, 0.2 g of fine powder is taken, put on a clean white paper and placed under the left nostril. Press and stop the right nostril, and inhale forcefully, and then the above-mentioned powder can be partially inhaled into the nasal cavity. The amount of solid powder inhaled each time was about 0.2 g, containing 18.6 mg of KCl (0.25 mmol KCl). In the case of using both left and right nostrils, about 37.3 mg of KCl (0.50 mmol KCl) would be inhaled in a single dose. Due to the high content of potassium salt, it absorbed water and dissolved in the nasal mucosa, and the salt concentration and osmotic pressure of the formed solution was high. The user had a slight dizziness, which basically disappeared after an hour.

### Embodiment 11

5.74 g of KCl was (molecular weight 74.55) dissolved in 500 mL of purified water (molar concentration 154 mmol/L, or 1.15% w/v) to form a clear solution, that is, a liquid inhalation formulation; then the solution was dispensed in a 50 mL push-type atomizing plastic bottle. When in use, the nozzles of the push-type spray plastic bottle are aimed at the left and right nostrils respectively, and the spray is pressed when taking a deep breath. The amount of liquid inhaled each time was about 0.2 mL, containing 2.3 mg of KCl (0.03 mmol KCl). In the case of using both left and right nostrils, about 4.6 mg of KCl (0.06 mmol KCl) would be inhaled in a single dose.

### Embodiment 12

100 g of potassium acetate (molecular weight 98.14) was dissolved in 100 mL of purified water (molar concentration 10000 mmol/L) to form a clear solution, that is, a liquid inhalation formulation; then the solution was dispensed in a 50 mL push-type atomizing plastic bottle. When in use, the nozzle of the push-type spray plastic bottle was aimed at the left and right nostrils respectively, and the spray is pressed when taking a deep breath. The amount of liquid inhaled each time was about 0.2 mL, containing 200 mg of KCl (2.04 mmol KCl). In the case of using both left and right nostrils, about 400 mg of KCl (4.08 mmol KCl) would be inhaled in a single dose. Due to the high concentration and osmotic pressure of the solution, the user had a slight dizziness, which basically disappeared after a period of time.

### Embodiment 13

269 g of potassium acetate (molecular weight 98.14) was dissolved in 100 mL of purified water (molar concentration 27,410 mmol/L) to form a clear solution, that is, a liquid inhalation formulation; then the solution was dispensed in a 50 mL push-type atomizing plastic bottle or an ultrasonic nebulizer. This solution can form a fine aerosol.

The following effect examples are real cases of using the inhalation formulation of the present disclosure. The subjects were patients all with hypertension for many years, aged between 40 and 70 years old. They took antihypertensive drugs for a long time but the control of blood pressure was not ideal. On an informed and voluntary basis, they accepted the nasal spray or the inhalation formulation of the present disclosure.

Effect Example 1

A man was found to have high blood pressure (95/145 mmHg) in 2015 at the age of 52. Antihypertensive drugs were taken orally at the beginning, but his blood pressure was not well controlled. Dizziness often occurred, and the quality of sleep was poor. The liquid inhalation formulation in Embodiment 1 was used according to the administration method of Embodiment 1, once in the morning and once in the evening. The amount of liquid inhaled each time was about 0.2 mL, containing 2.3 mg of KCl (0.03 mmol KCl). In the case of using both left and right nostrils, about 4.6 mg of KCl (0.06 mmol KCl) would be inhaled in a single dose. After using the nasal spray for one week, the subjective symptoms were improved and the blood pressure were decreased. Therefore, the oral antihypertensive drugs were stopped. In the follow-up time, only nasal spray or inhalation was used to control blood pressure (lasting more than 5 years and on-going). The subject's blood pressure was effectively controlled (below 85/135 mmHg) without using any other antihypertensive drugs. There were no adverse effects during this period.

Effect Example 2

A man, 57 years old, had anxiety and irritability due to poor sleep. The liquid inhalation formulation was used according to the administration method of Embodiment 1, twice in the morning at 7 am and 10 am respectively. Symptoms of anxiety and irritability disappeared at noon. The liquid inhalation formulation has been used in similar situations many times since then, with the same sedative effect being observed each time.

Effect Example 3

A man, 52 years old, had high blood pressure (95/145 mmHg). According to the administration method of Embodiment 7, the powder formulation was inhaled, once in the morning and once in the evening (the interval between administrations is one to three days), and the inhalation lasted for about ten minutes each time. After each use, the subject's blood pressure could be significantly reduced, generally down to 85/125 mmHg. There were no adverse effects during this period.

Effect Example 4

A man, 63 years old at the time, had a 10-year history of high blood pressure. He relied on oral antihypertensive drugs to control his condition, but sometimes the blood pressure still reached 95/155 mmHg. According to the administration method of Embodiment 3 (ultrasonic nebulizer), the liquid inhalation formulation of Embodiment 3 was used, once in the morning and once in the evening (the interval between administrations is one to three days), and the inhalation lasted for about ten minutes each time. The ultrasonic nebulizer can nebulize 6 mL of liquid every 30 minutes, which is equivalent to 69 mg of KCl (0.9 mmol). The inhalation lasted for about ten minutes each time, and about 23 mg of KCl (0.30 mmol KCl) was inhaled. After 10-20 minutes of each use, the subject's blood pressure could be significantly reduced, generally down to 85/135 mmHg. This subject has been taking liquid inhalation formulations as an effective means of assisting in controlling blood pressure in addition to oral medication for a year until now. There were no adverse effects during this period.

Effect Example 5

Zhang, 59 years old, had high blood pressure for 25 years. Before using nasal spray, he took half a tablet of Betaloc, 2 capsules of amlodipine, and 4 capsules of Luozhen every day. This patient used nasal spray as a supplemental means of blood pressure control beside oral antihypertensive medication.

According to the liquid inhalation formulation and usage method of Embodiment 11, the liquid inhalation formulation was used once in the morning and once in the evening every day, and it had been used continuously for 70 days. The subject measured his blood pressure with a sphygmomanometer every day and reported it. The blood pressure records are shown in FIG. 1. It can be seen from FIG. 1 that during the period of use, the subject's systolic and diastolic blood pressures both decreased, except there was a slight increase in blood pressure on the 6th day, which was due to the fact that the subject played mahjong for too long that day. During the entire use period, the subject did not have any other side effects except for occasional mild discomfort in the nasal cavity.

Effect Example 6

Zhang, 43 years old, had a 9-year history of hypertension. He took irbesartan and hydrochlorothiazide tablets 160 mg and 12.5 mg per day before using nasal spray. This patient used nasal spray as a supplemental means of blood pressure control beside oral antihypertensive medication.

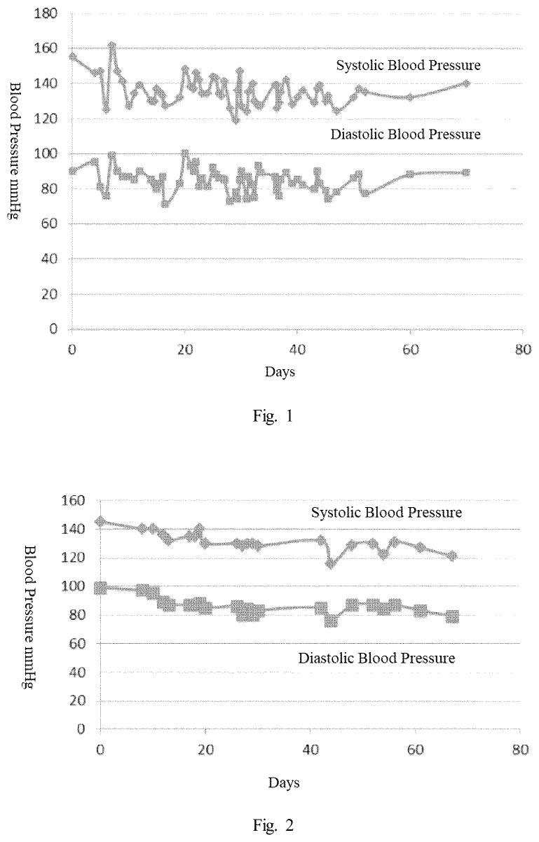

According to the liquid inhalation formulation and usage method of Embodiment 11, the liquid inhalation formulation was used once in the morning and once in the evening every day, and it had been used continuously for 70 days. After the nasal spray was used continuously for 45 days, the patient stopped taking the oral medicine for 3 days, then changed to taking the medicine every other day since the blood pressure was improved. The subject measured his blood pressure with a sphygmomanometer every day and reported it. The blood pressure records are shown in FIG. 2. It can be seen from FIG. 2 that during the period of use, the subjects' systolic and diastolic blood pressures both decreased. During the entire use period, the subject did not have any other side effects except for occasional mild discomfort in the nasal cavity.

Effect Example 7

Sun, 60 years old, had a 20-year history of hypertension. He took irbesartan and hydrochlorothiazide tablets 160 mg and 12.5 mg per day before using nasal spray. This patient used nasal spray as a supplemental means of blood pressure control beside oral antihypertensive medication.

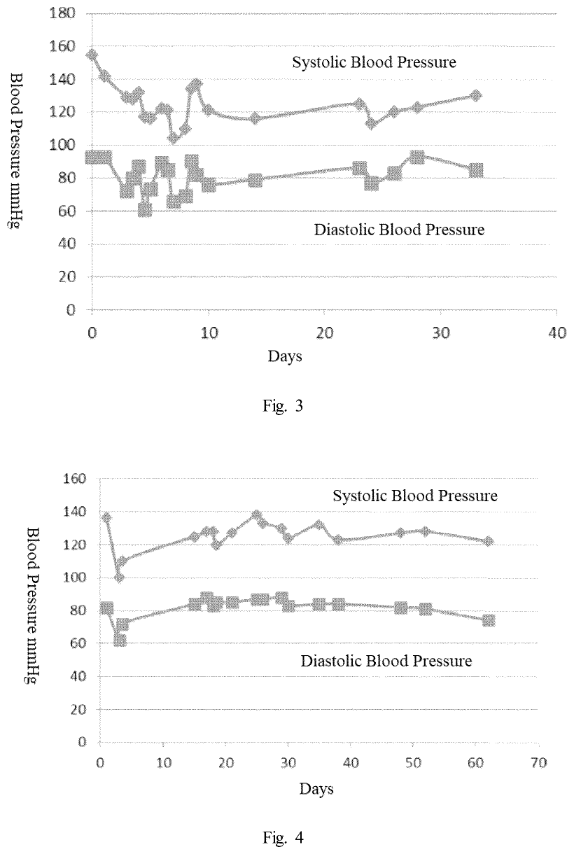

According to the liquid inhalation formulation and usage method of Embodiment 11, the liquid inhalation formulation was used once in the morning and once in the evening every day, and it had been used continuously for 35 days. From the 4th day of the nasal spray, the subject voluntarily discontinued the oral medication for 4 days. The subject measured his blood pressure with a sphygmomanometer every day and reported it. The blood pressure records are shown in FIG. 3. It can be seen from FIG. 3 that during the period of use, the subject's systolic and diastolic blood pressures both decreased. During the entire use period, the subject did not have any other side effects except for occasional mild discomfort in the nasal cavity.

Effect Example 8

Li, 59 years old, had a 15-year history of hypertension, during which he took medication intermittently. Amlodipine besylate tablets were taken daily before using the nasal spray, once a day and one tablet each time. This patient used nasal spray as a supplemental means of blood pressure control beside oral antihypertensive medication.

According to the liquid inhalation formulation and usage method of Embodiment 11, the liquid inhalation formulation was used once in the morning and once in the evening every day, and it had been used continuously for 60 days. From the 25th day of the nasal spray, the subject voluntarily discontinued the oral medication for 1 day, and the blood pressure rebounded on that day. Later on, he resumed amlodipine besylate tablets while using the nasal spray. The subject measured his blood pressure with a sphygmomanometer every day and reported it. The blood pressure records are shown in FIG. 4. It can be seen from FIG. 4 that during the period of use, the subject's systolic and diastolic blood pressures both decreased. During the entire use period, the subject did not have any other side effects except for occasional mild discomfort in the nasal cavity.

Although the specific implementations of the present disclosure have been described above, those skilled in the art should understand that these are only examples, and various changes or modifications can be made to these implementations without departing from the principle and essence of the present disclosure. Accordingly, the protection scope of the present disclosure is defined by the appended claims.

1-13. (canceled)

14. A liquid inhalation formulation, wherein
the liquid inhalation formulation consists of a potassium salt and water, with the molar concentration of potassium being 0.001 to 27,410 mmol/L;
or, the liquid inhalation formulation consists of a potassium salt, water, and a pharmaceutically acceptable additive, with the molar concentration of potassium being 0.001 to 27,410 mmol/L; the additive comprises one or more selected from a group consisting of a diluent, an acidity regulator, an isotonic regulator and a preservative.

15. The liquid inhalation formulation according to claim 14, wherein the potassium salt comprises one or more selected from a group consisting of potassium chloride, potassium acetate, potassium gluconate, potassium aspartate, potassium amino acid complex, potassium carbonate, potassium bicarbonate, potassium citrate, potassium dihydrogen citrate, potassium hydrogen citrate, potassium tartrate, potassium hydrogen tartrate, potassium hydroxide, potassium phosphate, potassium hydrogen phosphate, potassium dihydrogen phosphate, potassium sulfate, potassium bisulfate, potassium nitrate, potassium alginate and potassium sorbate.

16. The liquid inhalation formulation according to claim 14, wherein the molar concentration of potassium in the liquid inhalation formulation is 0.001 to 200 mmol/L, or 400 to 10,000 mmol/L.

17. The liquid inhalation formulation according to claim 14, wherein the liquid inhalation formulation is potassium chloride aqueous solution, potassium acetate aqueous solution, potassium carbonate aqueous solution, potassium bicarbonate aqueous solution, potassium tartrate aqueous solution, potassium hydrogen tartrate aqueous solution, potassium citrate aqueous solution or potassium hydrogen citrate aqueous solution.

18. A dry powder inhalation formulation, comprising a potassium salt and a drug carrier, and the content of the potassium salt is 0.01% to 100%, and the percentage is the mass percentage of the potassium salt in the dry powder inhalation formulation; the drug carrier comprises lactose.

19. The dry powder inhalation formulation according to claim 18, wherein the potassium salt comprises one or more selected from a group consisting of potassium chloride, potassium acetate, potassium gluconate, potassium aspartate, potassium amino acid complex, potassium carbonate, potassium bicarbonate, potassium citrate, potassium dihydrogen citrate, potassium hydrogen citrate, potassium tartrate, potassium hydrogen tartrate, potassium hydroxide, potassium phosphate, potassium hydrogen phosphate, potassium dihydrogen phosphate, potassium sulfate, potassium bisulfate, potassium nitrate, potassium alginate and potassium sorbate.

20. The dry powder inhalation formulation according to claim 18, wherein the content of the potassium salt in the dry powder inhalation formulation is 1% to 20%.

21. The dry powder inhalation formulation according to claim 18, wherein the dry powder inhalation formulation comprises potassium chloride and lactose; wherein the content of the potassium chloride is 2% to 10%, the percentage is the mass percentage of the potassium chloride in the dry powder inhalation formulation.

22. The dry powder inhalation formulation according to claim 18, wherein the average particle size of the dry powder inhalation formulation is 0.05 to 1,000 μm.

23. A method for treating hypertension or for keeping sedative in a subject in need thereof, comprising: administering a liquid inhalation formulation or a dry powder inhalation formulation to the oral cavity or respiratory tract of the subject, wherein the liquid inhalation formulation comprises a potassium salt and water, with the molar concentration of potassium being 0.001 to 27,410 mmol/L, the dry powder inhalation formulation comprising a potassium salt and a drug carrier, and the content of the potassium salt is 0.01% to 100%, and the percentage is the mass percentage of the potassium salt in the dry powder inhalation formulation.

24. The method according to claim 23, wherein the administration method for the liquid inhalation formulation comprises spraying, dripping or washing.

25. The method according to claim 23, wherein the administration method for the liquid inhalation preparation is that the supplementation of potassium ion is achieved continuously through breathing by the human body with continuous liquid spray in the environment.

26. The method according to claim 23, wherein in terms of potassium ions, the single dose of the liquid inhalation formulation is 0.01 to 0.5 mmol.

27. The method according to claim 23, wherein the administration method for the dry powder inhalation preparation is direct inhalation or use of a dry powder inhalation device;
or, the administration method for the dry powder inhalation preparation is that the supplementation of potassium ion is achieved continuously through breathing by the human body with continuous dry powder spray in the environment.

28. The method according to claim 23, wherein in terms of potassium ions, the single dose of the dry powder inhalation formulation is 0.01 to 0.5 mmol.
